(19) United States
(12) Patent Application Publication
(10) Pub. No.: US 2016/0271400 A1
(43) Pub. Date: Sep. 22, 2016

(54) Stamped Flange For Electric Feedthroughs With Integrated Grounding Pin

(71) Applicant: BIOTRONIK SE & Co. KG, Berlin (DE)

(72) Inventors: Daniel Kronmueller, Nuernberg (DE); Michael Arnold, Erlangen (DE); Lilli Fries, Stein (DE)

(21) Appl. No.: 15/057,398

(22) Filed: Mar. 1, 2016

Related U.S. Application Data
(60) Provisional application No. 62/135,716, filed on Mar. 20, 2015.

(51) Int. Cl. A61N 1/375; H01B 17/26

(52) U.S. Cl. CPC A61N 1/3754; H01B 17/26

(57) ABSTRACT

A feedthrough of an implantable medical electronic device, including an insulating body, a feedthrough flange surrounding the insulating body, and at least one connection element penetrating through the insulating body for the external connection of an electric or electronic component of the device, wherein the feedthrough flange has at least one pre-stamped and bent and/or folded and/or deep-drawn sheet metal part, in particular, formed of a titanium sheet or titanium alloy sheet, and the sheet metal part has an extension that is integrally formed in one piece and is angled relative to the plane of extension of the feedthrough and is formed as a ground connection surface.

### CROSS-REFERENCE TO RELATED APPLICATIONS

This patent application claims the benefit of co-pending U.S. Provisional Patent Application No. 62/135,716, filed on Mar. 20, 2015, which is hereby incorporated by reference in its entirety.

### TECHNICAL FIELD

The present invention relates to a feedthrough for an implantable electromedical device. Such a device typically comprises a device housing, in which electronic and electrical function units are housed, a device head having at least one electrode or a line connection, and a feedthrough arranged between the device housing and the device head for at least one electrical conductor element connecting the electrodes or the line connection to a function unit. A feedthrough of this type comprises a ceramic or glass insulating body, a feedthrough flange surrounding the insulating body, and at least one connection element penetrating through the insulating body for the external connection of an electrical or electronic component of the device. The present invention also relates to a method for producing such a feedthrough.

### BACKGROUND

Implantable devices of the above-mentioned type have long been used on a mass scale, in particular, as cardiac pacemakers or implantable cardioverters (especially defibrillators). However, said device may also be a less complex device, such as, for example, an electrode line or sensor line. Besides the use of feedthroughs in devices for heart therapy, feedthroughs are also used in cochlear implants.

Most implantable electromedical devices of practical significance are intended to deliver electrical pulses to excitable body tissue via suitably placed electrodes. In order to perform this function, electronic/electrical function units for generating the pulse and for suitably controlling the pulse generation are housed in the housing of the device, and electrodes or connections are provided directly on the device externally for at least one electrode line, in the distal end portion of which the electrodes are attached to the tissue for pulse transmission. The electronic/electrical function units in the device interior are to be connected to the outer electrodes or electrode line connections in such a way that ensures utterly and permanently reliable function under the special conditions of the implanted state. The connections or electrode lines can also be used to selectively measure electrical pulses and stimuli in the body of the patient and to record or evaluate these over a relatively long period of time in order to select an individually tailored therapy and to check the success of the treatment.

In particular, feedthroughs of which the main, insulating body consists substantially of ceramic or glass are known, wherein multilayer or multi-part superstructures have also been developed with use of metals or metal oxides and are used. Such known feedthroughs largely satisfy the requirements placed thereon with regard to, for example, hermeticity, biocompatibility, signal transmission and long-term stability.

In order to provide a ground potential for the electronic/electrical components and modules of the device, a connection to the metal housing thereof is established, specifically typically by a special ground connection means in the region of the feedthrough, particularly what is known as a grounding pin. With some types of known devices, a grounding pin, for example, made of niobium or Pt/Ir, is joined to the housing made of titanium by means of resistance welding. The electrical connection between the housing and the circuit board is established after welding or soldering the grounding pin on the circuit board and after welding the flange into the housing. Alternatively, the ground connection is produced by means of a pin that is mounted in a blind bore during assembly and is soldered to the flange in a high-temperature soldering process. The electrical connection through the housing is produced after soft soldering the feedthrough on the circuit board and after welding the flange into the housing.

In order to produce the ground connection in this way, an additional component is required. This has to be managed in the stockholding database, in the store, in the construction, etc. During the production process, this additional component may become confused with other components that have different dimensions. Since the grounding pin is not continuous, it is a shorter component part compared to other pins. Since the grounding pin is required only once per feedthrough, the number of said pins that is required is very low. Cost savings with regard to purchasing can only be attained with difficulty.

In order to contact the grounding pin and the flange, a separate joining process is necessary. This is a separate process for conventional bipolar and quadpolar feedthroughs. This can be implemented, for example, by means of a welding process, in which the grounding pin is electrically and conductively joined to the housing by means of resistance or laser welding. This process step requires appropriate documentation, validation or verification and must be subject to quality control.

Alternatively, the ground connection is produced in a manner integrated with the other contact elements by means of hard soldering. Here, there is the risk, however, that the joint is not optimally designed for the ground connection. In the event of the optical inspection of the feedthroughs, only the upper and lower side of the solder can be checked. In the case of normal joints, a two-sided check is sufficient to conclude whether the solder is sufficiently fused and distributed. In the case of ground connections, however, this conclusion cannot be drawn since only one-sided inspection is possible.

In order to be able to come to conclusions regarding the tensile strength or load-bearing capacity of a joined ground connection, additional tests are often necessary. Thus, microsections or a tensile test can reliably characterize the connection. Alternative destruction-free methods (for example, computer tomography) are very costly and play almost no role in practice.

The production and testing of a separate joint for ground contact requires additional process time. The fitting of the additional components and the testing or determination of the quality of the joint results in additional costs that increase the cost of the feedthrough. Since the grounding pin differs from the other pins, it interrupts the normal procedure in many process or test sequences and requires separate treatment. The separate process steps for the grounding pin must be clearly described in the documentation. The grounding pin must always be identified or described as such.

The increased requirements on reliability of the grounding pin require particular tests or a special design. By breaking the ground connection, the normal signal transmission is disrupted, but the device does not fail completely. The floating of the signals can lead to non-reproducible system errors and may not be diagnosed externally or may only be diagnosed with great difficulty.

International Publication No. WO 2013/122947 describes a feedthrough for an implantable medical device in which a cast feedthrough flange has an integrally cast tab arranged in the feedthrough plane for mounting a grounding pin.

The present invention is directed toward overcoming one or more of the above-mentioned problems.

### SUMMARY

An object of the present invention is to provide an improved feedthrough of an implantable electromedical device, with which, in particular, the necessary ground connection means is provided in a flexible, economical and durable manner. In particular, the ground connection of the feedthrough to the electronics will exceed the average service life of the implant whilst minimizing the outlay for production. Furthermore, a suitable method for producing such a feedthrough and also an improved implantable medical electronic device will be specified.

At least this object is achieved in a first device aspect by a feedthrough having the features of claim 1, and in accordance with a second device aspect by a device having the features of claim 12, and also in a method aspect by a method having the features of claim 9. Expedient developments of the inventive concept are specified in the respective dependent claims.

The present invention includes the concept of using a portion of the feedthrough flange directly as ground connection means, instead of an additional component part. Furthermore, the present invention includes the feature that the feedthrough flange has at least one pre-stamped and bent and/or folded and/or deep-drawn sheet metal part, in particular, formed from a titanium sheet or titanium alloy sheet. Lastly, it is proposed in linking the two aforementioned aspects for the sheet metal parts to have an extension molded integrally in one piece which is formed as a ground connection surface.

In a first embodiment, the present invention is applied in such a way that the integrally molded ground connection surface constitutes the only ground connection means of the feedthrough. Alternatively, in addition to the ground connection surface, a ground connection pin can be provided in a manner integrally bonded to said ground connection surface. Depending on the specific type of implantable device and the structural embodiment of the feedthrough thereof, one of the two variants may be preferred overall. Here, the electronics are connected in expedient embodiments by means of, for example, laser or resistance welding. To this end, a hybrid printed circuit board consisting of a rigid and a flexible circuit carrier is produced. The contact surfaces of the flexible circuit carrier can be contacted electrically at the individual pins or the grounding pin and joined thereto.

In further embodiments, the extension is formed with a mechanically stiffening profile contour, for example, a V-, U- or Z-profile, or a tube form. Due to the forming, the section modulus increases and prevents an undesirable distortion or deformation. The specific shaping of the stiffening contour(s) can be selected by a person skilled in the art from designs known per se for stiffening sheet metal parts, wherein contours other than the mentioned standard contours can also be used. The connection is made likewise to a hybrid printed circuit board according to the prior art by means of welding.

In a further embodiment, the free end of the extension is substantially cylindrical and has a surface that is enlarged compared to the longitudinal course of said extension. It is thus possible to provide better contact between the contact element that is to be attached and the enlarged surface of the extension. In particular, a sufficiently large contact surface to the device electronics is thus provided. Furthermore, it is easier to weld on the grounding pin, since the extension provides sufficient material for fusion and joining and also fixes and positions the counter-element reliably during the joining.

In order to improve the joinability, it is advantageous if the end face contains at least a small bore Φ0.1 mm for ventilation. The connection is made by welding a pin located on the circuit board. The counter-pin is fitted into the bushing shaped in the form of a sleeve and is electrically conductively welded.

In a further embodiment, the free end of the extension is substantially planar and has a width that is enlarged compared to the longitudinal course of said extension.

In particular, a sufficiently large connection surface of the flange extension to a connection surface of a printed circuit board (“PCB”) or another contact surface to the device electronics is provided. It may be advantageous to form this surface so is to be round, hexagonal or rectangular in accordance with the form of the land pattern of the printed circuit board and to match the size of the surfaces to one another.

With regard to the usual position of the corresponding printed circuit board, for example, in the housing of a cardiac pacemaker or implantable defibrillator, the widened and planar end of the extension, compared to the longitudinal extent thereof, can be folded approximately at right angles or bent accordingly so that the contact surfaces are arranged opposite one another or overlap over a large area, where possible. For contact, the two surfaces are joined together. This can be implemented, for example, by means of welding, soft soldering or hard soldering.

Further known forms of the end portion of the extension can also be advantageous for soft-soldered connections, for example, a forming into a gullwing, J-lead or SOP-like pin form. Notches or holes for ventilation of the weld gap are advantageous for welded connections.

In addition hereto or also independently hereof, the free end of the extension and/or a portion in the longitudinal course thereof can be provided with an additional (e.g., soft-solderable) contact material. Materials for such a contact material can be , for example, as follows, in particular: Cu, Ag, Au, Ni, Pd, Pt, Ir, Fe, or alloys containing these and, in particular, CuAg0.10, CuAg0.10P, CuTeP. In principle, all known methods can be used to apply the contact materials, such as , for example, plating, rolling, welding, hard soldering, sputtering, electroplating or seam welding. In particular, the production by means of vertical wire welding, resistance welding and horizontal, wire and profile section welding are particularly well suited in order to apply thin contact materials in a localized manner to strips or sheets in an automated process.

The application of the contact studs is typically a few μm to 100 μm, and the typical diameter can also vary in the range from 0.5 to 10 mm and can be brought into the desired form by embossing or tumbling.

Soft-solderable contact materials are advantageous for surface-mounting technology (“SMT”), especially on partial areas of the extension or over the entire region of the enlarged surface of the extension.

The feedthrough can be attached by the soft-solderable contact material on the extension by means of an SMT process directly during the printed circuit board assembly. The feedthrough can thus be placed on the printed circuit board already with other electronic components in automatic placement machines. The contact stud formed from an easily soft-solderable material is placed here directly in the solder paste on the printed circuit board (“PCB”). During soft soldering in the reflow process the solder paste is joined to the contact stud and forms an electrical contact. After soldering, the circuit board, inclusive of the feedthrough, can be tested as a whole and, in particular, standardized electrical and visual test systems can be used, for example, automatic optical inspections (“AOIs”), flying-probe tests (“FPTs”), in-circuit tests (“ICTs”), or the like.

In a further embodiment, the extension has at least two bending points in the longitudinal course in order to provide compressive and tensile resilience and/or in order to provide a length compensation reserve. Additional bending points of the extension can be used as resilient elements and can thus absorb forces and provide compensation, for example, during device assembly. A mechanical loading of the joint between the extension and circuit board can thus be minimized. Furthermore, it is possible to ensure that the ground connection breaks as the last pin in the event of mechanical loading, since the bending points act as a compensation element.

The compensation element can absorb and balance out changes in length or an axial offset. To this end, the length compensation reserve of the ground extension is selected such that the average length compensation is slightly above the normal pins, typically 0.05 mm. It is thus ensured that the extension reliably contacts the printed circuit board and the feedthrough in the slightly compressed state. If the feedthrough or the individual contacts are loaded by tension or compression, it is still ensured that the feedthrough does not fail. It is possible to balance out changes in length in the range of up to 0.2 mm, and to thus ensure that the ground connection is interrupted last of all signal connections.

Since with soft-solderable feedthroughs increased demands are placed on the coplanarity over all end faces of the pin ends or contact surfaces, it is particularly favorable if the extension can provide a length compensation reserve. The grounding pin is thus prevented from causing coplanarity breaches, and the likelihood of a coplanarity breach is reduced.

With regard to method features, the present invention includes the concept of, in a first step, pre-forming the sheet metal part comprising the extension as a sheet metal blank with a predetermined separation line, and removing the extension from the sheet metal blank along the predetermined separation line in a chronologically separate second step, in particular, immediately prior to the assembly of the feedthrough, and bringing the extension into position by forming.

This is then particularly advantageous if the production of the feedthrough flange and the assembly of the feedthrough take place at different production facilities. Then, the excess material can be easily broken off or removed during the assembly of the feedthrough and, until then, protects the ground connection surface and also the flange during transport and prior processes against a wedging or hooking of the parts. The thin grounding pin can thus be protected against damage and deformation. The required dimensional accuracy can be ensured in bulk material form, even with long periods of transport or pre-treatment processes (“cleaning”). Grooves or separating edges for alignment or orientation with the further-processing machine can be formed in the excess material. An automated further processing of the parts is thus possible very easily.

The efficiency of the production of flanges with integrated grounding pin can be further increased by using belts or rollers for production. The flange is separated out fully as far as the connecting webs. The connecting webs protect the flange and the grounding pin during transport against deformation and ensure a uniform supply into a further-processing facility. Once supplied into a facility or for further processing, the connecting web can be separated from the flange. A predetermined breaking point can be formed for this purpose in an end portion of the connecting web in one exemplary embodiment. This predetermined breaking point can be produced by forming a notch, at least a continuous recess in this region, or a selective tapering of the material. The supply of the stamped parts by rollers or belts is particularly economical for medium and large quantities.

In accordance with one embodiment of the inventive method, at least the extension of the sheet metal part, or a sheet metal used as starting material, is provided at least in regions with a polymeric or other organic protective layer as oxidation layer (e.g., “OSP”—organic surface protection). Known protective films (for example, Glicoat) can be deposited completely or selectively on the pin following the partial or complete separation. Typical layer thicknesses are 0.2 to 0.6 μm and, for example, consist of substituted imidazoles and/or triazoles. The protective films prevent the oxidation of the base material during storage, typically for a number of months, and pyrolyze immediately before or during the hard soldering or soft soldering process.

The proposed implantable device, which comprises a feedthrough having at least some of the above-described features, is formed, in particular, as a cardiac pacemaker or implantable cardioverter. However, other types of implantable medical devices can also be fitted with the feedthrough according to the present invention if these devices require a ground connection means, and if an at least bent and/or folded and/or deep-drawn sheet metal part can be used with these devices as part of the feedthrough flange.

On the one hand, the integrally molded extension alone can be provided in such a device as ground connection means of the electrical or electronic component and for this purpose can be connected directly in an integrally bonded manner to the component, in particular, a line carrier. Alternatively, the extension and an additional ground connection pin, which on the one hand is integrally bonded to the component, in particular, a printed circuit board or another circuit carrier, and on the other hand is integrally bonded to the extension, can be provided jointly as ground connection means. Although, in the case of a combination solution of this type, not all the advantages of the present invention attainable in principle can be achieved, this solution can provide a gain compared to known configurations, especially with regard to the reliability of the ground connection, but also with regard to certain technical simplifications.

In particular, one or more of the following advantages can be achieved with the present invention, at least in certain embodiments (as explained further above by way of example):
Since there are no transitions or material combinations at the transition between flange and ground connection, the thermal expansion is identical and, therefore, the component is not subject to thermal or mechanical loading.
The provision of the ground connection is possible in the technically simplest manner and thus economically, also due to the omission of test steps and corresponding documentation. As a result of the stamping, the flanges with an integrated ground connection surface can be produced very economically in high numbers. Since a number of cuts always have to be made, many flanges can be produced in parallel and, therefore, the throughput of the production rises. The assembly time of the feedthroughs reduces by the periods usually necessary for the steps of the supply and integration of pin, solder and solder pad. Due to the integration of soft-solderable materials on the head of the pin or in the semi-finished product of the sheet metal, assembly time can be saved in addition.
There are significant simplifications in the logistics of the parts required for production of an implantable device, including the associated purchasing and stockholding software.
Improvements with regard to the reliability of the device can be expected, since the number of possible error sources is significantly reduced compared to solutions that require additional parts. Due to the omission of a joint between ground connection and flange, no production faults, diffusion faults or corrosion problems can occur here either. The consistency of the material from flange to connection surface rules out thermoelectric effects at the transition points. DC voltage offsets can thus be ruled out systematically. Since no transitions or material combinations are provided at the transition between flange and ground connection surface, the thermal expansion is identical and, therefore, the component is not subject to thermal or mechanical stresses.
Due to the omission of the joint with at least two to three different materials, the typical framework and structural changes, intermetallic phases and particle range limits are spared.
Due to the use of a length compensation reserve, the coplanarity of the feedthrough can be improved and the failure of the implant under extreme vibration and shock loads can be minimized.

Further embodiments, features, aspects, objects, advantages, and possible applications of the present invention could be learned from the following description, in combination with the Figures, and the appended claims.

### DESCRIPTION OF THE DRAWINGS

Advantages and expedient features of the present invention will also emerge from the description of exemplary embodiments with reference to the Figures, in which:

FIG. 1 shows a schematic, partly cut illustration of an implantable electromedical device.

FIG. 2 shows a perspective illustration of a sheet metal part for producing a feedthrough flange according to an embodiment of the present invention.

FIGS. 3A-3C show schematic longitudinal sectional illustrations of feedthroughs according to further exemplary embodiments of the present invention.

FIG. 4 shows a schematic plan view of a sheet metal part of a feedthrough flange according to a further exemplary embodiment of the present invention.

FIGS. 5A and 5B show schematic perspective illustrations (detailed views) of feedthrough flanges according to further exemplary embodiments of the present invention.

FIG. 6 schematic perspective illustration of a finished feedthrough flange.

FIG. 7 schematic illustration of a stamped comb.

### DETAILED DESCRIPTION

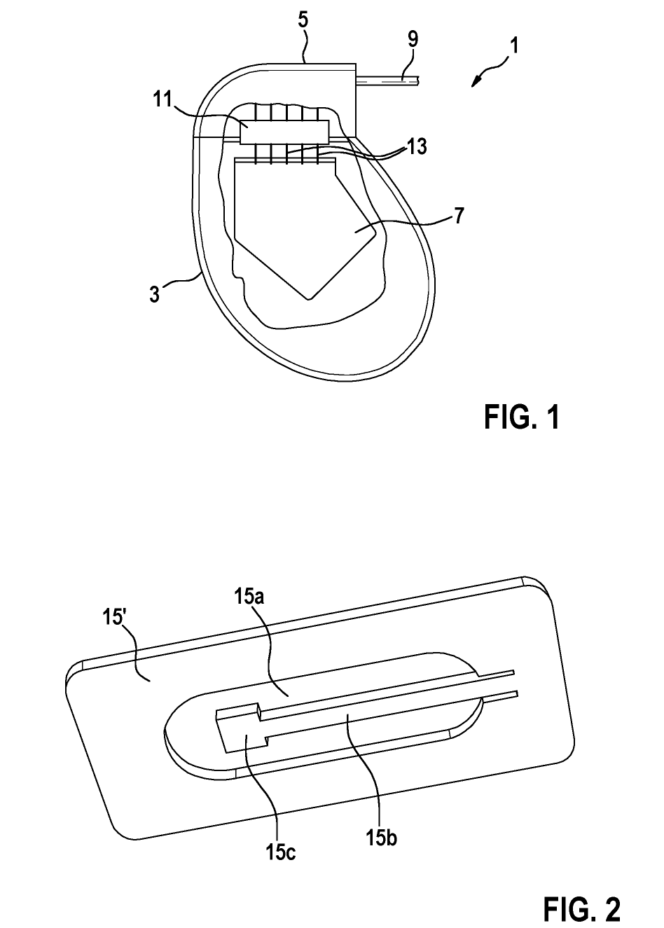

FIG. 1 shows a cardiac pacemaker 1 with a pacemaker housing 3 and a head part (header) 5, in the interior of which a printed circuit board (“PCB”) 7 is arranged in addition to other electronic components, an electrode line 9 being connected to the line connection (not shown) of said printed circuit board 7, which line connection is arranged in the header 5. A feedthrough 11 provided between the device housing 3 and the header 5 comprises a plurality of connection pins 13. The connection pins 13 are plugged at one end through a corresponding bore in the printed circuit board 7 and are soft-soldered thereto.

FIG. 2 shows a schematic perspective illustration of a sheet metal part 15′ that is stamped out from a sheet metal of a material suitable for producing a feedthrough flange and provides a feedthrough flange that can be produced easily and economically following forming steps. The sheet metal part 15′ has an approximately rectangular outer contour with rounded corners and a recess 15a, which is likewise approximately rectangular, with opposite semi-circular end regions, is stamped out internally.

An elongate rectangular extension 15b with widened, approximately square end portion 15c remains within this recess. Within the scope of the further forming steps of the sheet metal part 15′, the extension 15b is bent downwards approximately at right angles, and the end portion 15c can be bent, again approximately at right angles, in relation to this longitudinal course of the extension, such that the end portion is ultimately aligned again parallel to the plane of extension of the flange and, thus, parallel to the extension of a conductor or connection surface arranged typically in the housing of the device 1 (see FIG. 1). It can therefore be connected easily in an integrally bonded manner, in particular, soldered, to the connection surface.

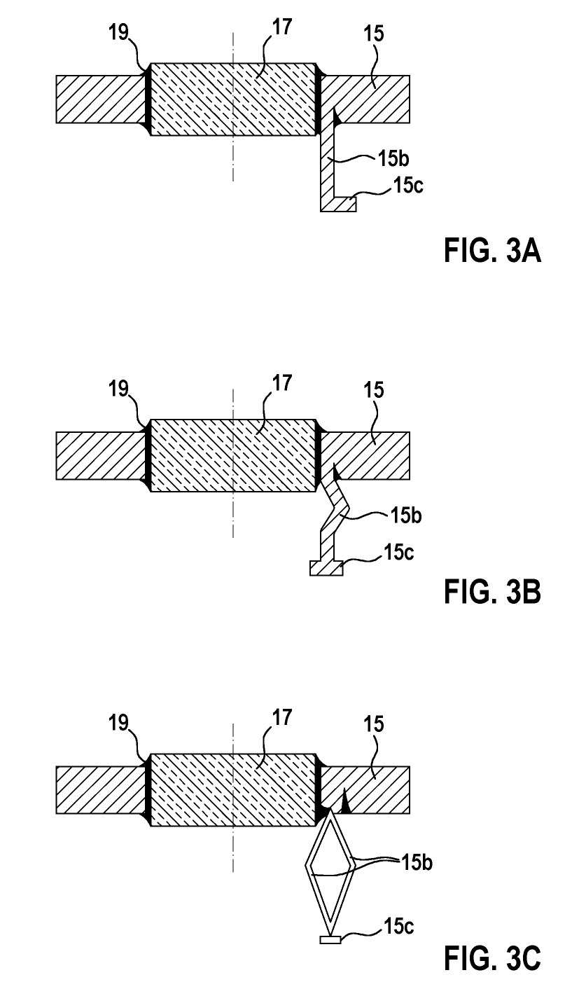

Such a state is shown in FIG. 3A as a schematic longitudinal sectional illustration. This figure, besides the feedthrough flange 15, also shows a main or insulating body 17 inserted therein. Typically, one or more connection pins for external signal connection of electronic/electrical components of the device are incorporated in said body 17 in the manner shown schematically in FIG. 1, however, such connection pins are not shown in FIG. 3A. A peripheral hard-soldered connection 19 around the outer edge of the main or insulating body 17 secures this body in the feedthrough flange 15 and at the same time ensures a hermetic seal, which also extends over incisions (not denoted separately) between the extension 15b and the surrounding flange material and, thus, also produces a closure and seal there. Here, formed edges or radii formed by the bending are covered and sealed by hard solder (for example, gold). Potential leakage paths in the region of the grounding pin are thus permanently closed by the normal hard soldering process (e.g., brazing). Leaks up to a limit value of 1e-11 mbar 1/s can be detected during normal leak detection.

FIGS. 3B and 3C show modifications in the shaping of the extension forming the ground connection surface of the implantable device, wherein, in spite of the different embodiment, the same reference signs as in FIGS. 2 and 3A are used. In the embodiment shown in FIG. 3B, three bend points are provided in the longitudinal course of the extension 15b that ensure a spring effect or a resilient yielding of the ground connection and, thus, an increased mechanical load-bearing capacity thereof. This is advantageous, in particular, in view of the fact that, in a device of the type discussed here, the ground connection should be interrupted last of all connections provided in the feedthrough under high mechanical stress and, therefore, no ‘floating’ signals are produced. These advantages can also be asserted with the embodiment according to FIG. 3C, in which the extension 15b is split in the longitudinal direction as far as the start of the end portion 15c and the two parts of said extension have been splayed apart in opposite directions during the forming process.

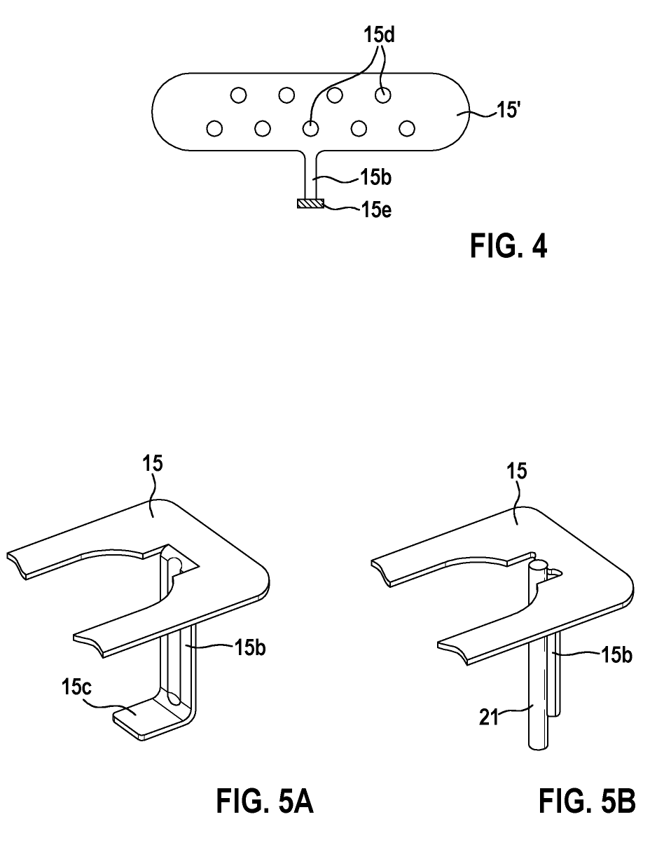

FIG. 4, again with use of the same reference signs as in FIGS. 2 to 3C, shows a further modified configuration. Here, the sheet metal part 15′ is provided with a plurality of smaller recesses 15d, in which thin insulating bodies are to be arranged later. The extension 15b providing the ground connection surface, together with the widened end portion 15c thereof, is therefore integrally molded to the outer contour of the flange 15. The extension is shown here still in the unformed state, but the end portion 15c thereof is already provided with a soft-solderable coating 15e in order to facilitate the subsequent step of connection to conductor or connection surfaces in the device.

Generally, the following is noted with regard to the production or processing steps of sheet metal parts for feedthrough flanges of the type shown in FIGS. 1 and 4.

If the production of the flange and the assembly of the feedthrough are performed at different facilities, it may be advantageous for the extension (e.g., the connection surface) and the flange to be manufactured with a predetermined breaking point or perforation and excess material. The excess material can thus be easily broken off or removed during assembly. It protects the extension and the flange during transport and previous processes against a wedging or hooking of the parts. The required dimensional accuracy of the parts can also be ensured for transport in the form of bulk material. Grooves or separating edges can be formed in the excess material for alignment or orientation with the further-processing machine. An automated further processing of the parts is thus possible very easily.

In order to increase the joinability of the pin to the circuit board, contact points can be applied to the pin already at semi-finished product stage. These contact points can be joined, for example, by means of welding or hard soldering. In the region of the pin head or pad, it is advantageous to join on the semi-finished product of the master sheet a surface made of nickel or copper or another material well suited for soft soldering prior to the separation. Here, the join point does not need to meet any increased demands on dimensional accuracy, since the surrounding region of the material will be separated in the subsequent process step by means of detachment.

It may be advantageous to seal surfaces of the extension, or also of the entire sheet metal part or coating thereof until further processing, with a polymer or an organic protective film (e.g., “OSP”—organic surface protection). Known protective films can be deposited completely or selectively on the pin following the partial or complete separation. Typical layer thicknesses are 0.2 to 0.6 μm and, for example, consist of substituted imidazoles and/or triazoles. The protective films prevent the oxidation of the base material during storage, typically for several months, and pyrolyze immediately before or during the hard soldering or soft soldering process.

FIG. 5A, in a sketched manner, shows a further modification, in which the extension 15b, serving as a ground connection surface of the device, is contoured in the cross section thereof in a V-shaped manner in order to stiffen said extension. However, the end portion 15c is also formed flat here in order to form a usable soldering surface. However, a special widening compared to the rest of the longitudinal course of the extension is not provided in this embodiment.

FIG. 5B shows a similar embodiment, but with semi-circular contouring instead of V-shaped contouring of the extension 15b and without folded or bent end portion. This is not required here since, in this configuration, a grounding pin 21 soldered to the extension 15b is provided as additional ground connection element. It is ultimately the end thereof that is soldered on a corresponding connection surface to a line carrier of the device. It is noted here that this embodiment at present is to be considered only for special applications of the present invention due to the technical and cost-based disadvantages of said embodiment. A special advantage of this combination is the creation of a large-surface and extraordinarily reliable mechanical and electrical connection between the ground connection element and the actual feedthrough flange.

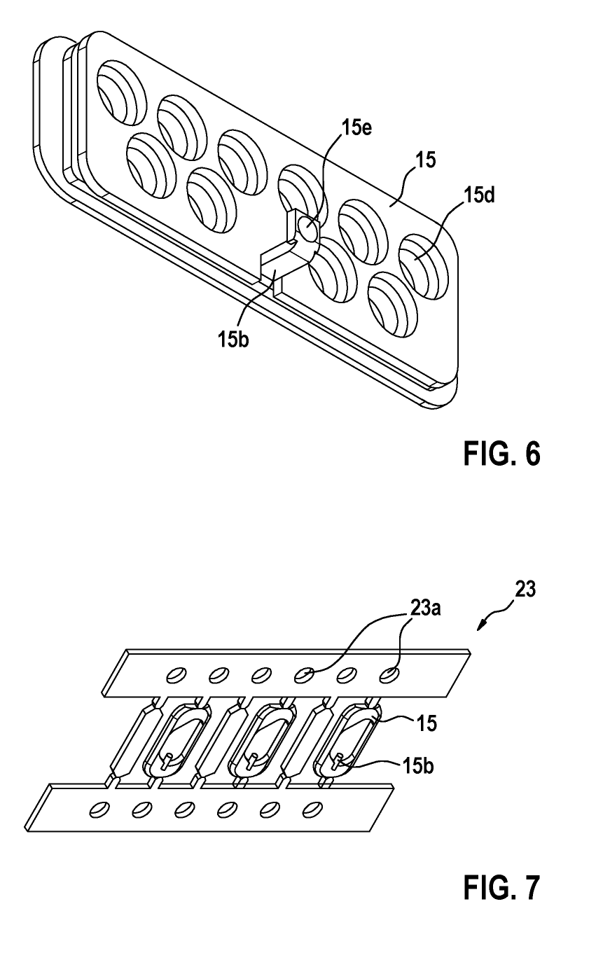

FIG. 6 shows the finished flange 15 of a ten-pole ICD feedthrough with a stamped grounding pin 15b. An insulation ceramic, and also a pin for signal conduction, can be inserted into each of the recesses 15d of the flange. A contact point 15e made of material that is well suited for soft soldering, here NiAg, which was joined to the base material by means of welding prior to the stamping of the flange contour, is located on the grounding pin. The grounding pin 15b was formed a number of times following the stamping of the flange, here the extension with the contact point 15e was established and bent into the position thereof. Following the joining of ceramic, pins and flange, the pins and the contact studs terminate on the recess in a coplanar manner, that is to say in a common plane. The permitted deviation is typically 0.1 mm.

FIG. 7, by way of example, shows a stamped comb 23 following production for ICD flanges with grounding pins 15b. The original material strip is oriented, guided and synchronized in the die by means of the holes 23a. The contours are stamped out, folded and, where appropriate, formed in steps. Following the production, the flanges 15 can be removed manually or in an automated manner from the punched comb 23 at the break edges. The grounding pin 15b can be formed or positioned in a second facility or, where appropriate, in a manner integrated in the process.

The embodiment of the present invention is also possible in a number of modifications of the examples shown here and aspects of the present invention discussed further above.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

I/we claim:

1. A feedthrough of an implantable medical electronic device, comprising:
an insulating body;
a feedthrough flange surrounding the insulating body; and
at least one connection element penetrating through the insulating body for the external connection of an electric or electronic component of the device,
wherein the feedthrough flange has at least one pre-stamped and bent and/or folded and/or deep-drawn sheet metal part formed of a titanium sheet or titanium alloy sheet, and
wherein the sheet metal part has an extension that is integrally formed in one piece and is angled relative to the plane of extension of the feedthrough and is formed as a ground connection surface.

2. The feedthrough as claimed in claim 1, wherein the integrally formed ground connection surface constitutes the only ground connection means of the feedthrough.

3. The feedthrough as claimed in claim 1, in which a ground connection pin is provided in addition to the ground connection surface and is connected thereto in an integrally bonded manner.

4. The feedthrough as claimed in claim 1, wherein the extension has a mechanically stiffening profile contour formed therein comprising a V-, U- or Z-profile or a tube form.

5. The feedthrough as claimed in claim 1, wherein a free end of the extension is substantially planar and is formed with an enlarged surface compared to the longitudinal course thereof.

6. The feedthrough as claimed in claim 1, wherein a free end of the extension and/or a portion in the longitudinal course thereof is provided with a soft-solderable coating.

7. The feedthrough as claimed in claim 1, wherein the extension has a plurality of bend points in the longitudinal course for providing compressive and tensile resilience and/or for providing a longitudinal compensation reserve.

8. The feedthrough as claimed in claim 1, wherein the feedthrough flange is joined from a plurality of pre-formed parts and comprises a multi-layer sheet metal composite, and the ground connection surface is formed from an individual part.

9. A method for producing a feedthrough as claimed in claim 1, wherein the sheet metal part comprising the extension is pre-formed in a first step as a sheet metal blank with a predetermined separation line and the extension is removed from the sheet metal blank along the predetermined separation line in a chronologically separate second step immediately prior to the assembly of the feedthrough.

10. A method for producing a feedthrough as claimed in claim 1, wherein at least the extension of the sheet metal part or a sheet metal serving as starting material is provided at least in regions with a protective layer, including a polymeric or other organic protective layer, as an oxidation layer.

11. The method as claimed in claim 10, wherein the protective layer is deposited as a thin layer.

12. An implantable medical device having a feedthrough as claimed in claim 1.

13. The device as claimed in claim 12, formed as a cardiac pacemaker or implantable cardioverter or cochlear implant.

14. The device as claimed in claim 12, wherein the integrally molded extension alone is provided as ground connection means of the electrical or electronic component and for this purpose is connected directly in an integrally bonded manner to the component including a line carrier.

15. The device as claimed in claim 12, wherein the extension and an additional ground connection pin, which on the one hand is integrally bonded to the component including a line carrier, and on the other hand is integrally bonded to the extension, are provided jointly as ground connection means of the component.
